(19) United States
(12) Patent Application Publication
(10) Pub. No.: US 2022/0211270 A1
(43) Pub. Date: Jul. 7, 2022

(54) SYSTEMS AND METHODS FOR GENERATING WORKSPACE VOLUMES AND IDENTIFYING REACHABLE WORKSPACES OF SURGICAL INSTRUMENTS

(71) Applicant: Intuitive Surgical Operations, Inc., Sunnyvale, CA (US)

(72) Inventors: Brandon D. Itkowtiz, San Jose, CA (US); Paul W. Mohr, Mountain View, CA (US)

(21) Appl. No.: 17/611,269

(22) PCT Filed: May 19, 2020
(86) PCT No.: PCT/US2020/033599

§ 371 Date: Nov. 15, 2021

Related U.S. Application Data
(60) Provisional application No. 62/852,128, filed on May 23, 2019.

(51) Int. Cl. A61B 5/00; A61B 5/11; A61B 34/10; G16H 40/67

(52) U.S. Cl. CPC A61B 5/0013; A61B 5/1127; A61B 34/10; A61B 2034/107; G06T 2207/10068; G06T 2207/30004; G16H 40/67

(57) ABSTRACT

A method comprises generating a workspace volume indicating an operational region of reach. The method further comprises referencing the workspace volume to an image capture reference frame of an image capture device, and the image capture device captures image data. The method further comprises determining a reachable workspace portion of the image data that is within the workspace volume. In some embodiments, the method further comprises determining an unreachable portion of the image data that is outside of the workspace volume. In other embodiments, the method further comprises displaying the reachable workspace portion of the image data without the unreachable portion of the image data. In still other embodiments, the method further comprises displaying a false graphic in place of the unreachable portion of the image data.

### CROSS-REFERENCE TO RELATED APPLICATIONS

This application claims priority to and the benefit of the filing date of U.S. Provisional Application No. 62/852,128, entitled “SYSTEMS AND METHODS FOR GENERATING WORKSPACE VOLUMES AND IDENTIFYING REACHABLE WORKSPACES OF SURGICAL INSTRUMENTS,” filed May 23, 2019, which is incorporated by reference herein in its entirety.

### FIELD

The present disclosure is directed to determining reachable workspaces of surgical instruments during surgical procedures and displaying kinematic limits of the surgical instruments with respect to a target patient anatomy.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide a user with a field of view within the patient anatomy.

Some minimally invasive medical tools may be teleoperated, otherwise remotely operated, or otherwise computer-assisted. During a surgical procedure, a surgeon may want to know the kinematic limits of the surgical instruments being used. It may also be helpful for the surgeon to visualize the limits and any changes in the kinematic limits in real time. This would allow the surgeon to perform the surgical procedure more efficiently and with less potential harm to the patient. Systems and methods are needed for continually visualizing kinematic limitations of surgical instruments during a surgical procedure. Additionally, systems and methods are needed that would allow a surgeon to determine the kinematic limits of a surgical instrument before making any incisions in a patient.

### SUMMARY

Embodiments of the invention are best summarized by the claims that follow the description.

Consistent with some embodiments, a method is provided. The method includes generating a workspace volume indicating an operational region of reach. The method further includes referencing the workspace volume to an image capture reference frame of an image capture device, and the image capture device captures image data. The method further includes determining a reachable workspace portion of the image data that is within the workspace volume.

Consistent with other embodiments, a method is provided. The method includes generating a first workspace volume indicating a first operational region of reach. The method further includes generating a second workspace volume indicating a second operational region of reach. The method further includes generating a composite workspace volume by combining the first workspace volume and the second workspace volume. The method further includes referencing the composite workspace volume to an image capture reference frame of an image capture device, and the image capture device captures image data. The method further includes determining a reachable workspace portion of the image data that is within the composite workspace volume.

Consistent with other embodiments, a method is provided. The method includes generating a workspace volume indicating an operational region of reach. The method further includes referencing the workspace volume to an image capture reference frame of an image capture device, and the image capture device captures image data. The method further includes determining a reachable workspace portion of the image data that is within the workspace volume. The method further includes based on the determined reachable workspace portion, determining an incision location of an instrument.

Consistent with other embodiments, a method is provided. The method includes generating a workspace volume indicating a region of a reach of an instrument. The method further includes generating a workspace volume indicating a region of a reach of an arm of a manipulating system. The method further includes referencing the workspace volume corresponding to the instrument to an image capture reference frame of an image capture device, and the image capture device captures image data. The method further includes determining a reachable workspace portion of the image data that is within the workspace volume corresponding to the instrument.

Other embodiments include corresponding computer systems, apparatus, and computer programs recorded on one or more computer storage devices, each configured to perform the actions of the methods.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A is a schematic view of a teleoperational medical system according to some embodiments.

FIG. 1B is a perspective view of a teleoperational assembly according to some embodiments.

FIG. 1C is a perspective view of a surgeon control console for a teleoperational medical system according to some embodiments.

FIG. 2A illustrates a side view of a workspace volume of an instrument according to some embodiments.

FIGS. 2B-2D each illustrate side views of a workspace volume of an instrument with the instrument in different orientations according to some embodiments.

FIG. 3A illustrates a front view of a workspace volume for each instrument in a medical system according to some embodiments.

FIG. 3B illustrates a side view of a composite workspace volume in a medical system according to some embodiments.

FIG. 3C illustrates a top view of a composite workspace volume in a medical system according to some embodiments.

FIG. 3D illustrates a side view of a composite workspace volume in a medical system overlaid on a model of a patient anatomy according to some embodiments.

FIG. 4A is an image of a left and right-eye endoscopic view of a patient anatomy according to some embodiments.

FIG. 4B is a depth buffer image of a model of a patient anatomy generated from endoscopic data from a left and right-eye endoscopic view of the patient anatomy according to some embodiments.

FIG. 4C is a reconstructed three-dimensional image of a model of a patient anatomy generated from a depth buffer image of the patient anatomy according to some embodiments.

FIG. 5 is an image of a perspective view of a composite workspace volume for each instrument in a medical system at a surgical site according to some embodiments.

FIG. 6A is an image of an endoscopic view of a model of a reachable portion of a patient anatomy according to some embodiments.

FIG. 6B is an image of an endoscopic view of a model of a reachable portion of a patient anatomy with a false graphic according to some embodiments.

FIG. 7A is an image of an endoscopic view with a color-coded grid indicating a reachable workspace portion overlaid on a model of a patient anatomy according to some embodiments.

FIG. 7B is an image of an endoscopic view with color-coded dots indicating a reachable workspace portion overlaid on a model of a patient anatomy according to some embodiments.

FIG. 7C is an image of an endoscopic view with contour lines indicating a reachable workspace portion overlaid on a model of a patient anatomy according to some embodiments.

FIG. 8A illustrates a method for generating a workspace volume according to some embodiments.

FIG. 8B illustrates a method for generating a workspace volume according to some embodiments.

FIG. 9 is an image of a perspective view of a workspace volume for each instrument in a medical system at a surgical site according to some embodiments.

FIG. 10 is an image of an endoscopic view with a three-dimensional surface patch overlaid on a model of a patient anatomy according to some embodiments.

Embodiments of the present disclosure and their advantages are best understood by referring to the detailed description that follows. It should be appreciated that like reference numerals are used to identify like elements illustrated in one or more of the figures for purposes of illustrating but not limiting embodiments of the present disclosure.

### DETAILED DESCRIPTION

In the following description, specific details describe some embodiments consistent with the present disclosure. Numerous specific details are set forth in order to provide a thorough understanding of the embodiments. It will be apparent to one skilled in the art, however, that some embodiments may be practiced without some or all of these specific details. The specific embodiments disclosed herein are meant to be illustrative but not limiting. One skilled in the art may realize other elements that, although not specifically described, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances well known methods, procedures, components, and circuits have not been described in detail so as not to unnecessarily obscure aspects of the embodiments.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various special device positions and orientations. The combination of a body's position and orientation define the body's pose.

Similarly, geometric terms, such as “parallel” and “perpendicular” are not intended to require absolute mathematical precision, unless the context indicates otherwise. Instead, such geometric terms allow for variations due to manufacturing or equivalent functions.

In addition, the singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. The auxiliary verb “may” likewise implies that a feature, step, operation, element, or component is optional.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy), and performing procedures on human or animal cadavers. Further, these techniques can also be used for surgical and nonsurgical medical treatment or diagnosis procedures.

Further, although some of the examples presented in this disclosure discuss teleoperational robotic systems or remotely operable systems, the techniques disclosed are also applicable to computer-assisted systems that are directly and manually moved by operators, in part or in whole.

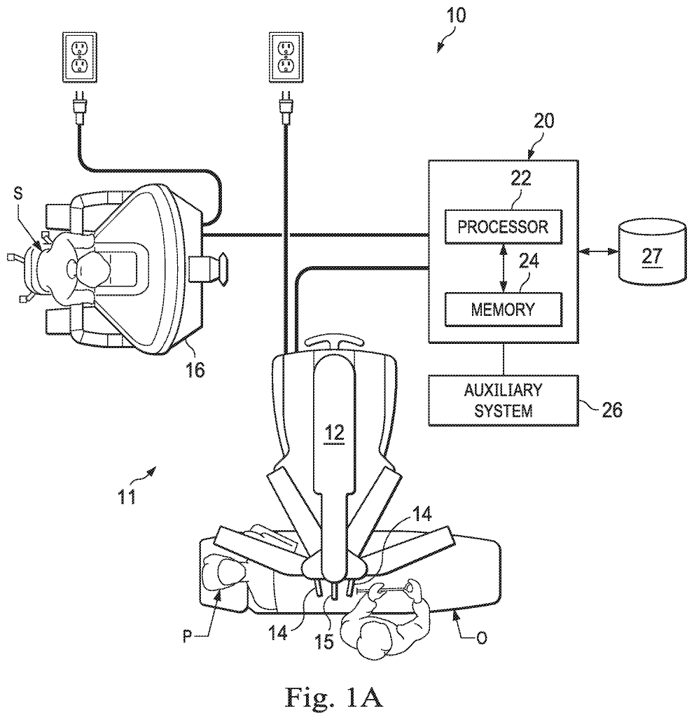

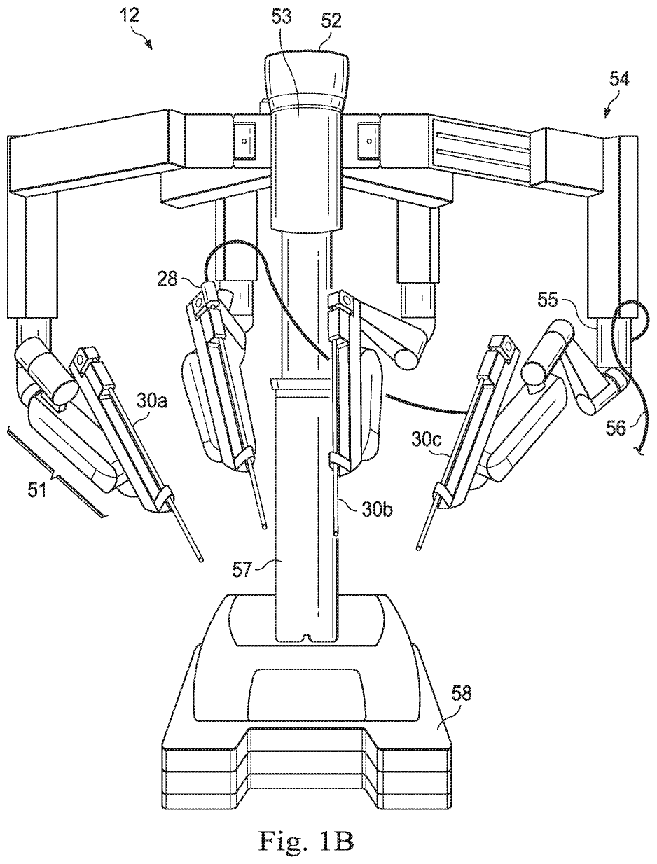

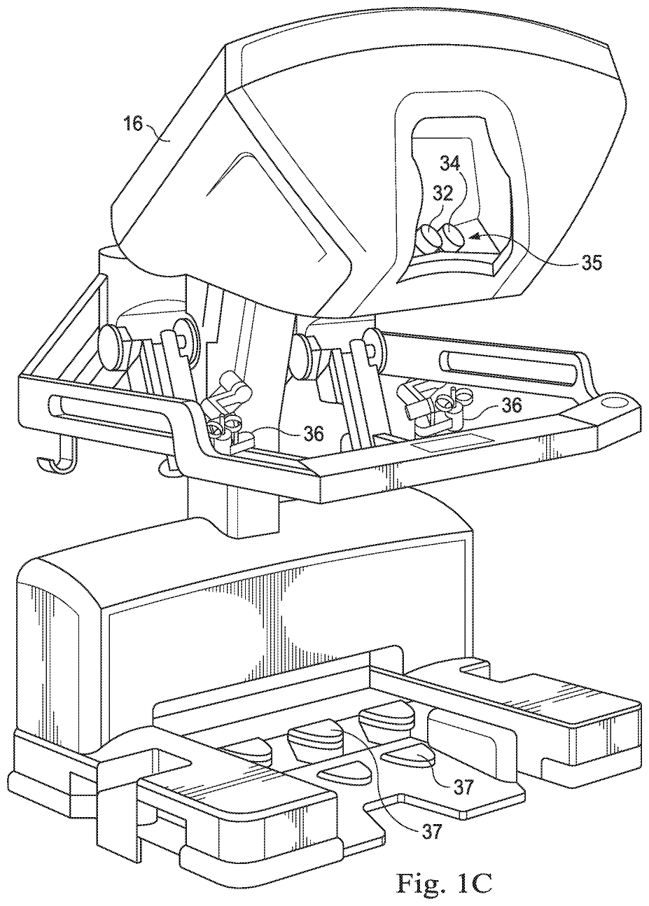

Referring now to the drawings, FIGS. 1A, 1B, and 1C together provide an overview of a medical system 10 that may be used in, for example, medical procedures including diagnostic, therapeutic, or surgical procedures. The medical system 10 is located in a medical environment 11. The medical environment 11 is depicted as an operating room in FIG. 1A. In other embodiments, the medical environment 11 may be an emergency room, a medical training environment, a medical laboratory, or some other type of environment in which any number of medical procedures or medical training procedures may take place. In still other embodiments, the medical environment 11 may include an operating room and a control area located outside of the operating room.

In one or more embodiments, the medical system 10 may be a teleoperational medical system that is under the teleoperational control of a surgeon. In alternative embodiments, the medical system 10 may be under the partial control of a computer programmed to perform the medical procedure or sub-procedure. In still other alternative embodiments, the medical system 10 may be a fully automated medical system that is under the full control of a computer programmed to perform the medical procedure or sub-procedure with the medical system 10. One example of the medical system 10 that may be used to implement the systems and techniques described in this disclosure is the da Vinci® Surgical System manufactured by Intuitive Surgical, Inc. of Sunnyvale, Calif.

As shown in FIG. 1A, the medical system 10 generally includes an assembly 12, which may be mounted to or positioned near an operating table O on which a patient P is positioned. The assembly 12 may be referred to as a patient side cart, a surgical cart, or a surgical robot. In one or more embodiments, the assembly 12 may be a teleoperational assembly. The teleoperational assembly may be referred to as, for example, a manipulating system and/or a teleoperational arm cart. An instrument system 14 and an endoscopic imaging system 15 are operably coupled to the assembly 12. An operator input system 16 allows a surgeon S or other type of clinician to view images of or representing the surgical site and to control the operation of the medical instrument system 14 and/or the endoscopic imaging system 15.

The medical instrument system 14 may comprise one or more medical instruments. In embodiments in which the medical instrument system 14 comprises a plurality of medical instruments, the plurality of medical instruments may include multiple of the same medical instrument and/or multiple different medical instruments. Similarly, the endoscopic imaging system 15 may comprise one or more endoscopes. In the case of a plurality of endoscopes, the plurality of endoscopes may include multiple of the same endoscope and/or multiple different endoscopes.

The operator input system 16 may be located at a surgeon's control console, which may be located in the same room as operating table O. In some embodiments, the surgeon S and the operator input system 16 may be located in a different room or a completely different building from the patient P. The operator input system 16 generally includes one or more control device(s) for controlling the medical instrument system 14. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same degrees of freedom as the medical instrument(s) of the medical instrument system 14 to provide the surgeon with telepresence, which is the perception that the control device(s) are integral with the instruments so that the surgeon has a strong sense of directly controlling instruments as if present at the surgical site. In other embodiments, the control device(s) may have more or fewer degrees of freedom than the associated medical instruments and still provide the surgeon with telepresence. In some embodiments, the control device(s) are manual input devices that move with six degrees of freedom, and which may also include an actuatable handle for actuating instruments (for example, for closing grasping jaw end effectors, applying an electrical potential to an electrode, delivering a medicinal treatment, and actuating other types of instruments).

The assembly 12 supports and manipulates the medical instrument system 14 while the surgeon S views the surgical site through the operator input system 16. An image of the surgical site may be obtained by the endoscopic imaging system 15, which may be manipulated by the assembly 12. The assembly 12 may comprise endoscopic imaging systems 15 and may similarly comprise multiple medical instrument systems 14 as well. The number of medical instrument systems 14 used at one time will generally depend on the diagnostic or surgical procedure to be performed and on space constraints within the operating room, among other factors. The assembly 12 may include a kinematic structure of one or more non-servo controlled links (e.g., one or more links that may be manually positioned and locked in place, generally referred to as a set-up structure) and a manipulator. When the manipulator takes the form of a teleoperational manipulator, the assembly 12 is a teleoperational assembly. The assembly 12 includes a plurality of motors that drive inputs on the medical instrument system 14. In an embodiment, these motors move in response to commands from a control system (e.g., control system 20). The motors include drive systems which when coupled to the medical instrument system 14 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 14 may include end effectors having a single working member such as a scalpel, a blunt blade, an optical fiber, or an electrode. Other end effectors may include, for example, forceps, graspers, scissors, or clip appliers.

The medical system 10 also includes a control system 20. The control system 20 includes at least one memory 24 and at least one processor 22 for effecting control between the medical instrument system 14, the operator input system 16, and other auxiliary systems 26 which may include, for example, imaging systems, audio systems, fluid delivery systems, display systems, illumination systems, steering control systems, irrigation systems, and/or suction systems. A clinician may circulate within the medical environment 11 and may access, for example, the assembly 12 during a set up procedure or view a display of the auxiliary system 26 from the patient bedside. In some embodiments, the auxiliary system 26 may include a display screen that is separate from an operator input system 16 (see FIG. 1C). In some examples, the display screen may be a standalone screen that is capable of being moved around the medical environment 11. The display screen may be orientated such that the surgeon S and one or more other clinicians or assistants may simultaneously view the display screen.

Though depicted as being external to the assembly 12 in FIG. 1A, the control system 20 may, in some embodiments, be contained wholly within the assembly 12. The control system 20 also includes programmed instructions (e.g., stored on a non-transitory, computer-readable medium) to implement some or all of the methods described in accordance with aspects disclosed herein. While the control system 20 is shown as a single block in the simplified schematic of FIG. 1A, the control system 20 may include two or more data processing circuits with one portion of the processing optionally being performed on or adjacent the assembly 12, another portion of the processing being performed at the operator input system 16, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one embodiment, the control system 20 supports wireless communication protocols such as Bluetooth, IrDA, HomeRF, IEEE 802.11, DECT, and Wireless Telemetry.

The control system 20 is in communication with a database 27 which may store one or more clinician profiles, a list of patients and patient profiles, a list of procedures to be performed on said patients, a list of clinicians scheduled to perform said procedures, other information, or combinations thereof. A clinician profile may comprise information about a clinician, including how long the clinician has worked in the medical field, the level of education attained by the clinician, the level of experience the clinician has with the medical system 10 (or similar systems), or any combination thereof.

The database 27 may be stored in the memory 24 and may be dynamically updated. Additionally or alternatively, the database 27 may be stored on a device such as a server or a portable storage device that is accessible by the control system 20 via an internal network (e.g., a secured network of a medical facility or a teleoperational system provider) or an external network (e.g., the Internet). The database 27 may be distributed throughout two or more locations. For example, the database 27 may be present on multiple devices which may include the devices of different entities and/or a cloud server. Additionally or alternatively, the database 27 may be stored on a portable user-assigned device such as a computer, a mobile device, a smart phone, a laptop, an electronic badge, a tablet, a pager, and other similar user devices.

In some embodiments, the control system 20 may include one or more servo controllers that receive force and/or torque feedback from the medical instrument system 14. Responsive to the feedback, the servo controllers transmit signals to the operator input system 16. The servo controller(s) may also transmit signals instructing assembly 12 to move the medical instrument system(s) 14 and/or endoscopic imaging system 15 which extend into an internal surgical site within the patient body via openings in the body. Any suitable conventional or specialized servo controller may be used. A servo controller may be separate from, or integrated with, assembly 12. In some embodiments, the servo controller and assembly 12 are provided as part of a teleoperational arm cart positioned adjacent to the patient's body.

The control system 20 can be coupled with the endoscopic imaging system 15 and can include a processor to process captured images for subsequent display, such as to a surgeon on the surgeon's control console, or on another suitable display located locally and/or remotely. For example, where a stereoscopic endoscope is used, the control system 20 can process the captured images to present the surgeon with coordinated stereo images of the surgical site. Such coordination can include alignment between the opposing images and can include adjusting the stereo working distance of the stereoscopic endoscope.

In alternative embodiments, the medical system 10 may include more than one assembly 12 and/or more than one operator input system 16. The exact number of assemblies 12 will depend on the surgical procedure and the space constraints within the operating room, among other factors. The operator input systems 16 may be collocated or they may be positioned in separate locations. Multiple operator input systems 16 allow more than one operator to control one or more assemblies 12 in various combinations. The medical system 10 may also be used to train and rehearse medical procedures.

FIG. 1B is a perspective view of one embodiment of an assembly 12 which may be referred to as a patient side cart, surgical cart, teleoperational arm cart, or surgical robot. The assembly 12 shown provides for the manipulation of three surgical tools 30a, 30b, and 30c (e.g., medical instrument systems 14) and an imaging device 28 (e.g., endoscopic imaging system 15), such as a stereoscopic endoscope used for the capture of images of the site of the procedure. The imaging device may transmit signals over a cable 56 to the control system 20. Manipulation is provided by teleoperative mechanisms having a number of joints. The imaging device 28 and the surgical tools 30a-c can be positioned and manipulated through incisions in the patient so that a kinematic remote center is maintained at the incision to minimize the size of the incision. Images of the surgical site can include images of the distal ends of the surgical tools 30a-c when they are positioned within the field-of-view of the imaging device 28. The imaging device 28 and the surgical tools 30a-c may each be therapeutic, diagnostic, or imaging instruments.

The assembly 12 includes a drivable base 58. The drivable base 58 is connected to a telescoping column 57, which allows for adjustment of the height of arms 54. The arms 54 may include a rotating joint 55 that both rotates and moves up and down. Each of the arms 54 may be connected to an orienting platform 53. The arms 54 may be labeled to facilitate trouble shooting. For example, each of the arms 54 may be emblazoned with a different number, letter, symbol, other identifier, or combinations thereof. The orienting platform 53 may be capable of 360 degrees of rotation. The assembly 12 may also include a telescoping horizontal cantilever 52 for moving the orienting platform 53 in a horizontal direction.

In the present example, each of the arms 54 connects to a manipulator arm 51. The manipulator arms 51 may connect directly to a medical instrument, e.g., one of the surgical tools 30a-c. The manipulator arms 51 may be teleoperatable. In some examples, the arms 54 connecting to the orienting platform 53 may not be teleoperatable. Rather, such arms 54 may be positioned as desired before the surgeon S begins operation with the teleoperative components. Throughout a surgical procedure, medical instruments may be removed and replaced with other instruments such that instrument to arm associations may change during the procedure.

Endoscopic imaging systems (e.g., endoscopic imaging system 15 and imaging device 28) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image based endoscopes have a “chip on the tip” design in which a distal digital sensor such as a one or more charge-coupled device (CCD) or a complementary metal oxide semiconductor (CMOS) device store image data. Endoscopic imaging systems may provide two- or three-dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle, and shaft all rigidly coupled and hermetically sealed.

FIG. 1C is a perspective view of an embodiment of the operator input system 16 at the surgeon's control console. The operator input system 16 includes a left eye display 32 and a right eye display 34 for presenting the surgeon S with a coordinated stereo view of the surgical environment that enables depth perception. The left and right eye displays 32, 34 may be components of a display system 35. In other embodiments, the display system 35 may include one or more other types of displays. In some embodiments, image(s) displayed on the display system 35 may be separately or concurrently displayed on a display screen of the auxiliary system 26.

The operator input system 16 further includes one or more input control devices 36, which in turn cause the assembly 12 to manipulate one or more instruments of the endoscopic imaging system 15 and/or the medical instrument system 14. The input control devices 36 can provide the same degrees of freedom as their associated instruments to provide the surgeon S with telepresence, or the perception that the input control devices 36 are integral with said instruments so that the surgeon has a strong sense of directly controlling the instruments. To this end, position, force, and tactile feedback sensors (not shown) may be employed to transmit position, force, and tactile sensations from the medical instruments, e.g., the surgical tools 30a-c or the imaging device 28, back to the surgeon's hands through the input control devices 36. Input control devices 37 are foot pedals that receive input from a user's foot. Aspects of the operator input system 16, the assembly 12, and the auxiliary systems 26 may be adjustable and customizable to meet the physical needs, skill level, or preferences of the surgeon S.

During a medical procedure performed using the medical system 10, the surgeon S or another clinician may want to know the available reach of one or more medical instruments (e.g., the surgical tools 30a-c or the imaging device 28). Knowing and visualizing the instrument reach may allow the clinicians to better plan a surgical procedure, including locating patient incision locations and positioning manipulator arms. During a surgical procedure, knowledge and visualization of the instrument reach may allow the surgeon to determine whether or which tools may be able to access target tissue or whether the tool, manipulator arms, and/or incision locations should be repositioned. Below are described systems and methods that may allow a clinician to determine the kinematic limitations of the surgical tools 30a-c and/or the imaging device 28 to assist with procedure planning and to prevent unexpectedly encountering those kinematic limitations during the surgical procedure.

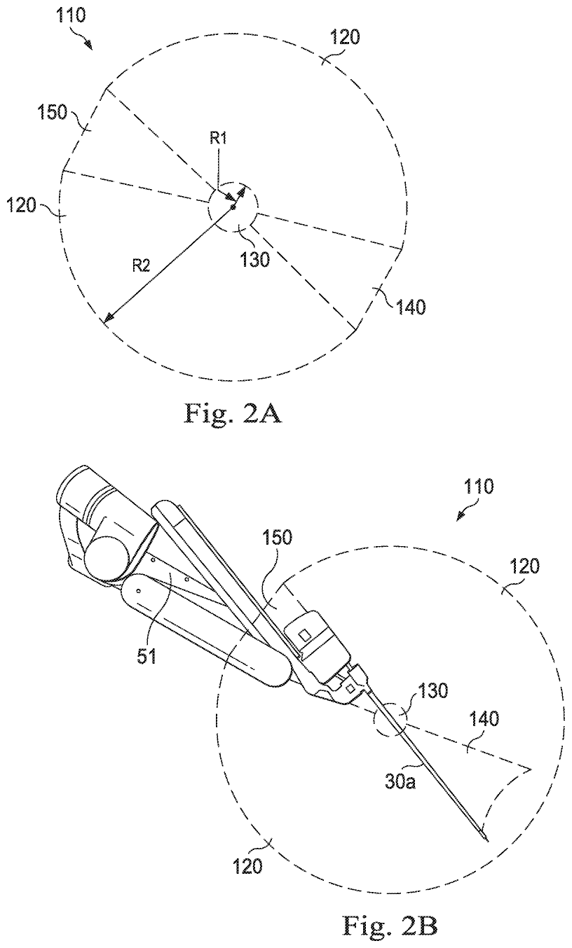

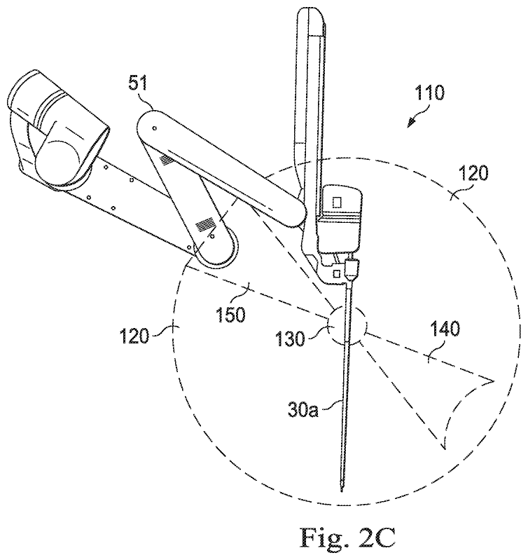

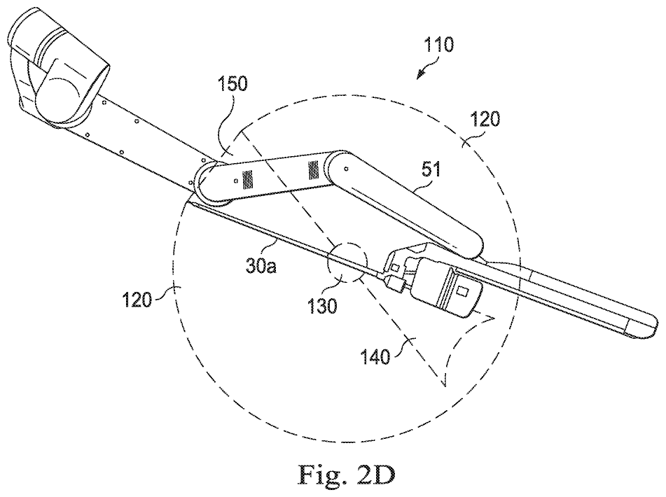

The various embodiments described below provide methods and systems that allow the surgeon S to more easily determine the kinematic limitations (e.g., a reachable workspace) of each of the surgical tools 30a-c and of the imaging device 28. In one or more embodiments, the display system 35 and/or the auxiliary systems 26 may display an image of a workspace volume (e.g., the workspace volume 110 in FIG. 2A) overlaid on a model of a patient anatomy in the field of view of the imaging device 28. The reachable workspace portion indicates the limits of a reach of one or more of the surgical tools 30a-c and/or the imaging device 28. Being able to view the reachable workspace portion may assist the surgeon S in determining the kinematic limitations of each of the surgical tools 30a-c and/or the imaging device 28 with respect to one or more internal and/or external portions of the patient anatomy.

FIG. 2A illustrates a side view of a workspace volume 110 of an operational region of reach according to some embodiments. The operational region of reach includes a region of reach of an instrument 30a. The operational region of reach may also include a region of reach of the manipulator arm 51. Additionally, the operational reach may include a region of reach of the arm 54. In some embodiments, the region of reach of the manipulator arm 51 defines the region of reach of the instrument 30a. Additionally, the region of reach of the arm 54 may define the region of reach of the manipulator arm 51. Therefore, the region of reach of the arm 54 may define the region of reach of the instrument 30a by defining the region of reach of the manipulator arm 51. The workspace volume 110 may be defined by any one or more of the region of reach of the instrument 30a, the region of reach of the manipulator arm 51, or the region of reach of the arm 54.

The workspace volume 110 includes a reachable workspace portion 120. The reachable workspace portion 120 of the workspace volume 110 illustrates a range of a reach of the instrument 30a, for example the range of reach of the distal end effector of the instrument 30a. As discussed above, the instrument 30a may move in six degrees of freedom (DOF)—three degrees of linear motion and three degrees of rotational motion. The motion of the instrument 30a may be driven and constrained, at least in part, by the movement of the manipulator arm 51 to which it attached. The workspace volume 110 also includes portions 130, 140, 150 that are not within reach of the instrument 30a. The unreachable portion 130 surrounds a remote center of motion the instrument 30. In some embodiments, the workspace volume 110 is a three-dimensional (3D) spherical volume. In other embodiments, the workspace volume 110 may be a cylindrical volume, a conical volume, or any other shape corresponding to the range of motion of the instrument. An inner radius R1 of the workspace volume 110 is determined by an insertion range of the instrument 30a. For example, the inner radius R1 may be determined by a minimum insertion limit of the instrument 30a. R1 may also be the radius of the unreachable portion 130. An outer radius R2 of the workspace volume 110 is also determined by the insertion range of the instrument 30a. For example, the outer radius R2 may be determined by a maximum insertion limit of the instrument 30a. In several examples, the unreachable portions 140, 150 are three dimensional conical volumes. All or portions of the workspace volume 110 be displayed as 2D or 3D imaging on the display system 35 and/or on a display screen of one or more systems of the auxiliary systems 26, as will be described below.

FIGS. 2B-2D each illustrate side views of the workspace volume 110 of the instrument 30a with the instrument 30a in different orientations according to some embodiments. Alternatively, the instrument may be one of the surgical tools 30b, 30c, or the instrument may be the imaging device 28. As shown in FIG. 2B, the instrument 30a may be arranged in a pitch-back pose. As shown in FIG. 2C, the instrument 30a may be arranged in an upright pose. As shown in FIG. 2D, the instrument 30a may be arranged in a pitch-forward pose. The poses of the instrument 30a in FIGS. 2B-2D may track the movement of the manipulator arm 51 to which the instrument 30a is attached. Rotational movement of the arm 51 allows the instrument 30 to access the full three-dimensional volume of the reachable workspace portion 120, including the volume located above the portions 140, 150.

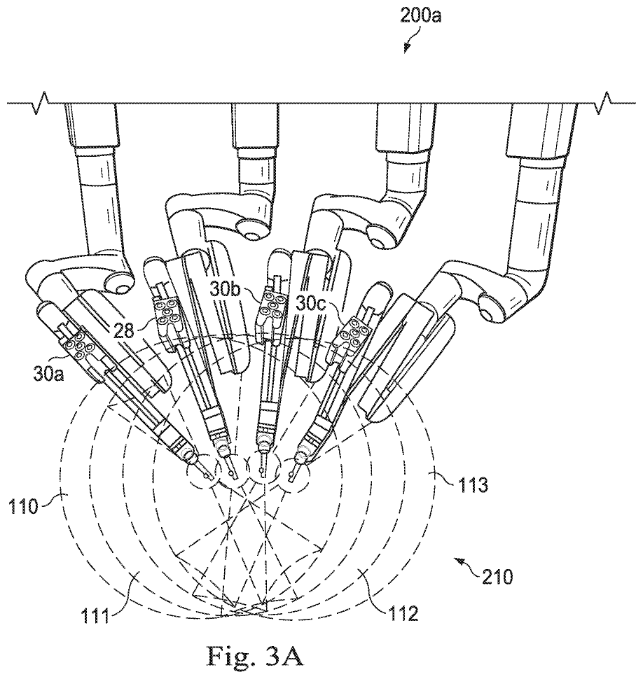

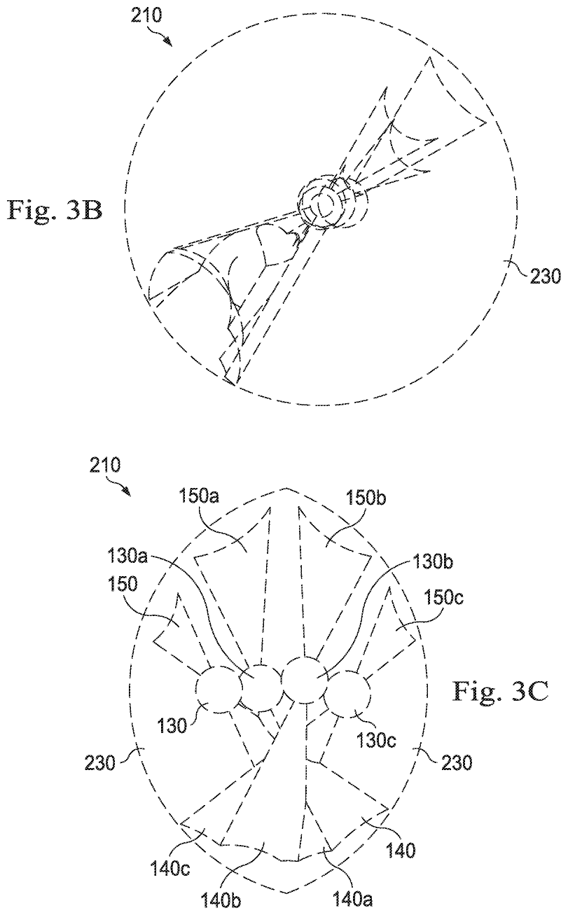

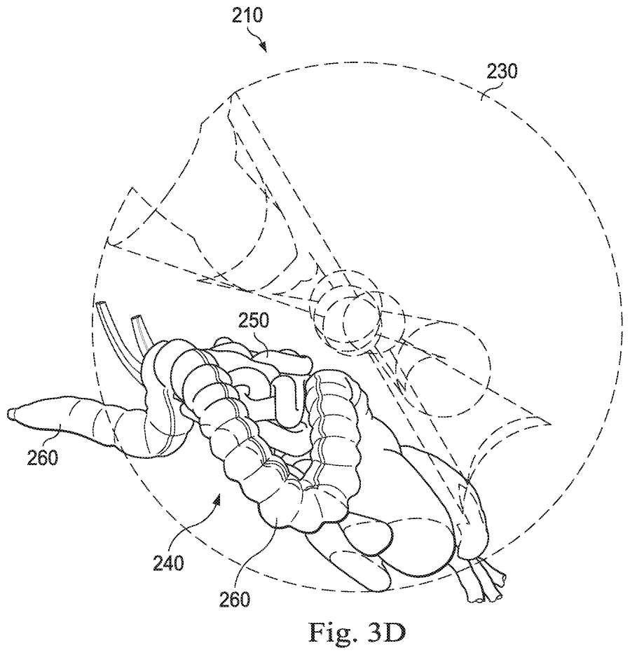

FIG. 3A illustrates a front view of a composite workspace volume 210 comprising the workspace volumes for each instrument 28, 30a-c in the medical system 10. More specifically, the composite workspace volume 210 includes the workspace volume 110 associated with instrument 30a, a workspace volume 111 associated with instrument 28, a workspace volume 112 associated with instrument 30b, and a workspace volume 113 associated with instrument 30c. In some embodiments, a workspace volume 210 includes a workspace volume for one or less than all of the instruments in the medical system 10. The amount of overlap between the workspace volumes depends on the proximity of each instrument in relation to every other instrument being used in the surgical procedure. In examples where the instruments are close together, such as in the embodiment of FIG. 3A, the workspace volumes for each of the instruments may significantly overlap each other. In examples where the instruments are spaced apart, the workspace volumes for each of the instruments may only slightly overlap each other. In other embodiments, the workspace volume for each of the instruments may not overlap each other at all and the composite workspace volume may include a plurality of discrete workspace volumes.

FIG. 3B illustrates a side view of the composite workspace volume 210. The composite workspace volume 210 includes a reachable workspace portion 230 that is reachable by one or more of the instruments 28, 30a-c. The composite workspace volume 210 also includes portions unreachable by one or more of the instruments 28, 30a-c. For example and as shown in FIG. 3C, portions 130, 140, 150 are unreachable by instrument 30a; portions 130a, 140a, 150a are unreachable by instrument 28; portions 130b, 140b, 150b are unreachable by instrument 30b; and portions 130c, 140c, 150c are unreachable by instrument 30c. The workspace volumes 110-113 can be combined into the composite workspace volume 210 using a constructive solid geometry (CSG) intersection operation. The CSG operation can be performed by the control system 20 and/or one or more systems of the auxiliary systems 26. In some embodiments, the surgeon S may toggle between views of the composite workspace volume 210 and a view of the workspace volume for each instrument 28, 30a-c, which will be discussed in further detail below. Being able to toggle among views of the workspace volumes 210 and the discrete volumes 110-113 may improve the surgeon's understanding of the abilities and constraints of each instrument or the set of instruments together.

FIG. 3C illustrates a top view of the composite workspace volume 210. As shown in FIG. 3C, the unreachable portions 140, 140a, 140b, 140c, 150, 150a, 150b, 150c for the instruments 28, 30a-c are subtracted from the workspace volume 210 leaving the reachable workspace portion 230. The reachable workspace portion 230 illustrates the volume which at least one of the instruments 28, 30a-c can reach. Accordingly, the outer boundary of the reachable workspace portion 230 of the composite workspace volume 210 is defined by the reachable workspace portion of the instrument with the greatest kinematic range. For example, if the instrument 30a has the longest reach out of the other instruments, then the reachable workspace portion 230 will be limited to the reach of the instrument 30a. In alternative embodiments, the reachable workspace portion may be defined as the volume that all of the instruments 28, 30a-c can reach. Thus, in this alternative embodiment, the instrument with the shortest reach may define the outer boundary of the reachable workspace portion.

FIG. 3D illustrates the composite workspace volume 210 and a patient anatomy 240 registered to a common coordinate system. The co-registration of the volume 210 and the patient anatomy generate an overlap that allows unreachable portions of the anatomy 240 to be identified. The patient anatomy 240 includes a reachable portion 250 and unreachable portions 260. The reachable portion 250 of the patient anatomy 240 includes portions of the patient anatomy 240 that are within the reachable workspace portion 230. The unreachable portion 260 of the patient anatomy 240 includes portions of the patient anatomy 240 that are outside of the reachable workspace portion 230. The portions of the patient anatomy 240 that are reachable versus unreachable will vary based on the placement of the instruments 28, 30a-c, a position of the arms 51 (see FIG. 1B), a patient size, the particular patient anatomy of interest 240, etc.

The workspace volume 210 either alone or registered with the patient anatomy 240 may be modeled and presented as a composite for viewing on the display system 35 or the auxiliary system 26. As discussed above, in several embodiments, the surgeon S can toggle between different views of the reachable workspace portion 230 or the individival reachable workspace portions (e.g., the reachable workspace portion 120). In other words, the surgeon S may view the reachable workspace portion for each instrument independently or in composite. This may allow the surgeon S to determine which instruments cannot reach a particular location. In other examples, the surgeon S may view on a display screen the reachable workspace portion of a workspace volume of a single-port robot when the surgeon S moves an entry guide manipulator to relocate a cluster of instruments included in the single-port robot. In other examples, the surgeon S may view a cross-section of the reachable workspace portion (e.g., the reachable workspace portion 120) at the current working distance of the instrument (e.g., the instrument 30a). In such examples, the surgeon S may view which portions of the patient anatomy 240 are within the reach of the instrument 30a in a particular plane, which may be parallel to a plane of the endoscopic view. In several embodiments, the surgeon S may view the reachable workspace portion 230 from a third-person view, rather than from the endoscopic view of the instrument 28. This may allow the surgeon S to visualize the extent of the reach of the instrument 30a, for example. In such embodiments, the surgeon S may toggle between the endoscopic view and the third-person view.

In other alternative embodiments, the reachable workspace portion of each instrument 28, 30a-c may be determined based on potential interactions/collisions between the arms 51. In such embodiments, the unreachable portions of the workspace volume, such as the workspace volume 110, for example, is determined based on physical interference that may occur between the arms 51. The workspace volume for each instrument 28, 30a-c is computed as a distance field. Therefore, for each instrument 28, 30a-c the closest distance between the surface of each arm 51 and all neighboring surfaces of each other arm 51 may be used to determine the reachable workspace volume. In some embodiments, an isosurface extraction method (e.g., marching cubes) may be used to generate a surface model of the unobstructed workspace of each arm 51. In some embodiments, the distance field is computed by sampling a volume around a tip of each instrument 28, 30a-c based on the position of each instrument 28, 30a-c. Then, inverse kinematics of each arm 51 may be simulated to determine the pose of each arm 51 at every candidate position for the tip of each instrument 28, 30a-c. Based on the simulated poses of each arm 51, the distance field, i.e., the closest distance between the surface of each arm 51 and all neighboring surfaces of each other arm 51, may be computed. From the computed distance field, a volumetric distance field may be produced that represents locations on the surface of each arm 51 where collisions between the arms 51 would occur. In several embodiments, the volumetric distance field is transformed into the endoscopic reference frame. For any image of the model of the patient anatomy 240 from the viewpoint of the imaging device 28, the volumetric distance field may be displayed as a false graphic in the image. In some examples, the false graphic indicates portions of the patient anatomy 240 that are unreachable by one or more of the instruments 28, 30a-c due to a collision that would occur between the arms 51.

In some embodiments, the reachable workspace volumes for each instrument 28, 30a-c may be displayed on the display system 35 and/or on a display screen of one or more systems of the auxiliary systems 26 before an incision is made in the patient P by one or more of the instruments 28, 30a-c. In other embodiments, the reachable workspace volume for each instrument 28, 30a-c may be displayed on the display system 35 and/or on a display screen of one or more systems of the auxiliary systems 26 before the instruments 28, 30a-c are installed on their corresponding arms 51. In still other alternative embodiments, the reachable workspace portion of each instrument 28, 30a-c may be determined based on potential interactions/collisions between the arms 54. In some embodiments, the reachable workspace portion of each instrument 28, 30a-c may be determined based on potential interactions/collisions between both the arms 51 and the arms 54.

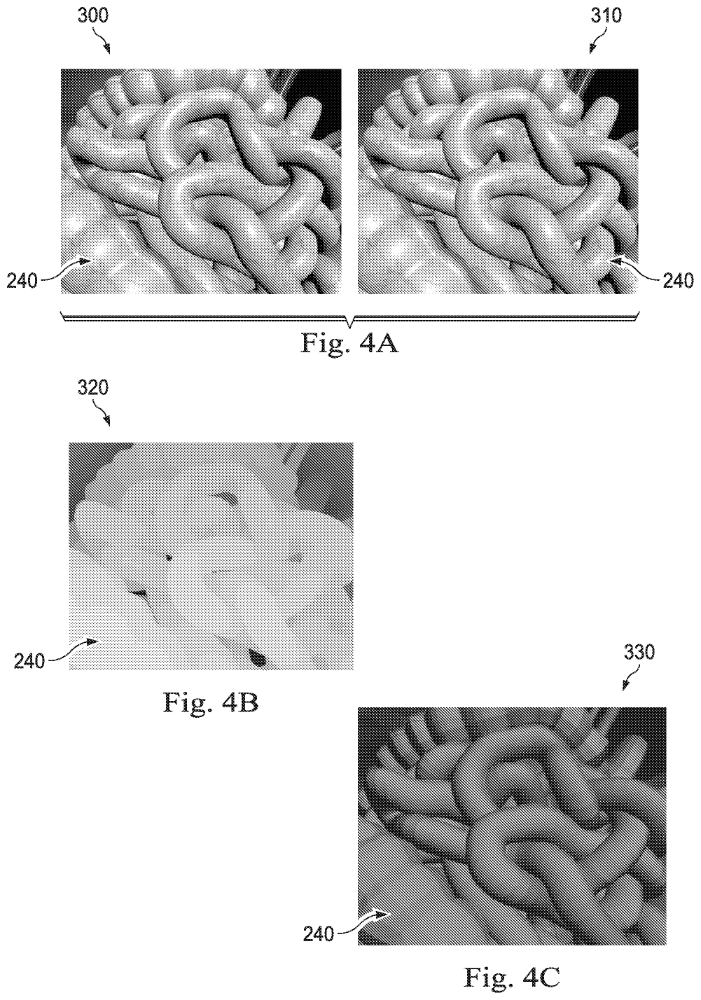

Composite views of the reachable workspace volume with views of endoscopic views of the patient anatomy (e.g. views obtained by the imaging instrument 28), may allow the clinician to visualize the boundaries of the workspace volume and the reach of one or more or of the instruments in at the work site. Stereoscopic composite views may be particularly useful, allowing the viewer to visualize the three-dimensional nature of the workspace volume, the patient anatomy, and the workspace boundaries. FIG. 4A illustrates an image 300 of a left-eye endoscopic view of the patient anatomy 240 and image 310 of a right-eye endoscopic view of the patient anatomy 240 according to some embodiments. The image 300 (which may include captured endoscopic data) is a left-eye image taken by a left camera eye of the imaging device 28. Some or all of the endoscopic data may be captured by the left camera eye of the imaging device 28. The image 310 (which may include captured endoscopic data) is a right-eye image taken by a right camera eye of the imaging device 28. Some or all of the endoscopic data may be captured by the right camera eye of the imaging device 28. The images 300, 310 each illustrate the patient anatomy 240 as viewed from an endoscopic reference frame, which may also be referred to as an image capture reference frame. The endoscopic reference frame is a reference frame at a distal tip of the imaging device 28. Therefore, the surgeon S can view the patient anatomy 240 from the point of view of the left and right eye cameras of the imaging device 28. As discussed in further detail below, the composite workspace volume 210 (and/or one or more of the workspace volumes 110) is referenced to the endoscopic reference frame.

FIG. 4B is a depth buffer image 320 of a model of the patient anatomy 240 generated from endoscopic data from a left and right-eye endoscopic view of the patient anatomy 240 according to some embodiments. In some embodiments, the control system 20 and/or one or more systems of the auxiliary systems 26 combines the left eye image 300 and the right eye image 310 to generate the depth buffer image 320. FIG. 4C is a reconstructed three-dimensional image 330 of a model of the patient anatomy 240 generated from the depth buffer image 320 of the patient anatomy 240 according to some embodiments. In some embodiments, the control system 20 and/or one or more systems of the auxiliary systems 26 generates the reconstructed 3D image 330 from the depth buffer image 320.

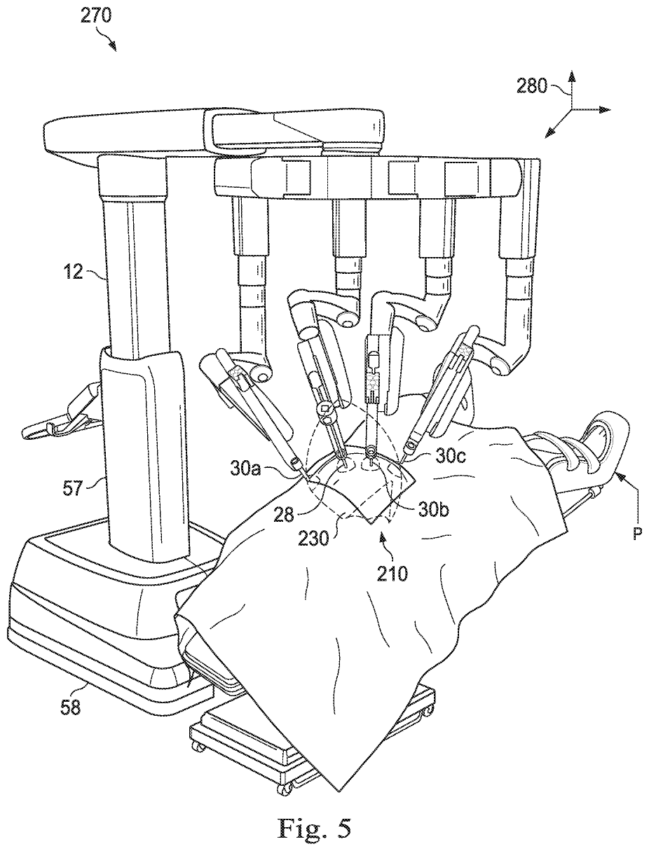

FIG. 5 is a perspective view of a system workspace 270 in which the patient P (which includes patient anatomy 240) and the assembly 12 are located. The system workspace 270 and the workspace volume 210 are registered to a common coordinate frame 280. As shown in FIG. 5, some sections of the reachable workspace portion 230 are external to the body of the patient P and some sections of the reachable workspace portion 230 (not shown) are internal to the body of the patient P.

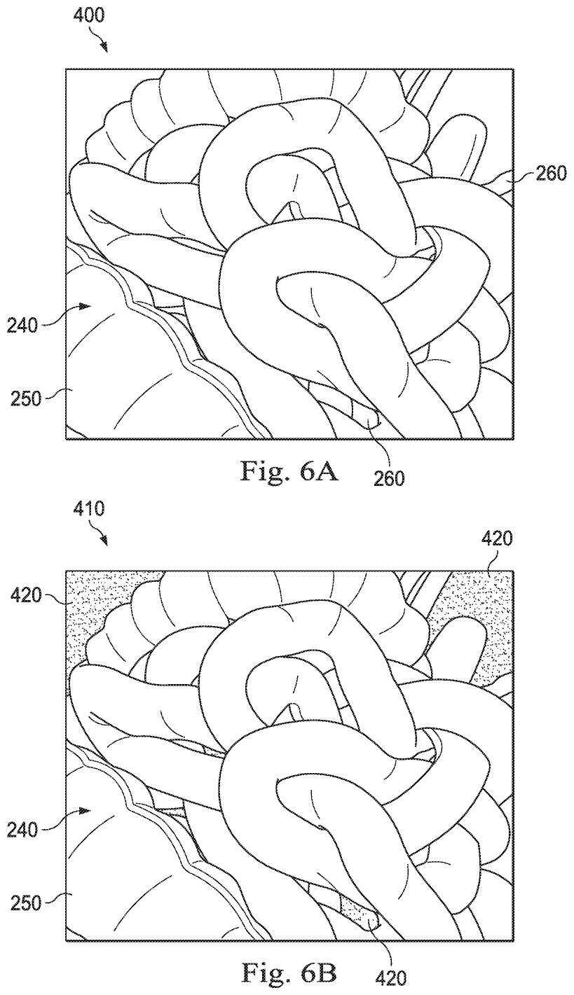

FIG. 6A is an image 400 of an endoscopic view of a model of the patient anatomy 240 according to some embodiments. The image 400 is an image from the endoscopic view of the imaging device 28. In some embodiments, the image 400 may be the reconstructed three-dimensional image 330 of a model of the patient anatomy 240 generated from the depth buffer image 320. The image 400 includes the reachable portion 250 and the unreachable portion 260 of the patient anatomy 240. FIG. 6B is an image 410 of an endoscopic view of a model of the patient anatomy 240 with a false graphic 420 according to some embodiments. The image 410 is an image from the endoscopic view of the imaging device 28. In some embodiments, the image 410 may be the reconstructed three-dimensional image 330 of a model of the patient anatomy 240 generated from the depth buffer image 320. The image 410 includes the reachable portion 250 of the patient anatomy 240. The image 410 also includes the false graphic 420 which may occlude the unreachable portion 260 of the patient anatomy 240 or otherwise graphically distinguish the unreachable portion 260 from the reachable portion 250.

In some embodiments, the reachable workspace portion 230 is overlaid on an image of the patient anatomy 240 to allow the surgeon S to see which portions of the patient anatomy 240 are within the reach of the instruments 28, 30a-c. As shown in FIG. 6B, the false graphic 420 is included in the image 410. In some examples, the false graphic 420 may be displayed in place of the unreachable portion 260 of the patient anatomy 240. In some embodiments, the false graphic 420 may include a color hue, a color saturation, an illumination, a surface pattern, cross-hatching, or any other suitable graphic to distinguish the reachable portion 250 of the patient anatomy 240 from the unreachable portion 260 of the patient anatomy 240. In other embodiments, the reachable portion 250 of the patient anatomy 240 is displayed in the image 410, and the unreachable portion 260 of the patient anatomy 240 is not displayed in the image 410.

In some embodiments, the false graphic 420 is displayed in the image 410 when one or more of the arms 51 and/or the arms 54 of the assembly 12 are moved within the operating room (see FIG. 1A) to adjust the workspace occupied by the assembly 12. In some instances, the arms 54, 51 are manually adjusted. Each of the arms 54, 51 includes a control mode that allows the operator to adjust the spacing of the arms 54, 51 relative to each other and relative to the patient P in order to adjust redundant degrees of freedom to manage the spacing between the arms 54, 51. The spacing between the arms 54, 51 may be managed while the pose of the tip of the instruments 28, 30a-c is maintained. In other instances, each of the arms 54, 51 includes an additional control mode that optimizes the positions of the arms 54, 51. In this additional control mode, the arms 54, 51 are positioned relative to each other to maximize the reach of the instruments 28, 30a-c during the surgical procedure. When either or both of these control modes are active, the false graphic 420 may be displayed in the image 410. Being able to visualize the reachable portion 250 of the patient anatomy 240 assists with optimizing the positions of the arms 54, 51 in the workspace, which aids in optimizing the reach of the instruments 28, 30a-c during the surgical procedure.

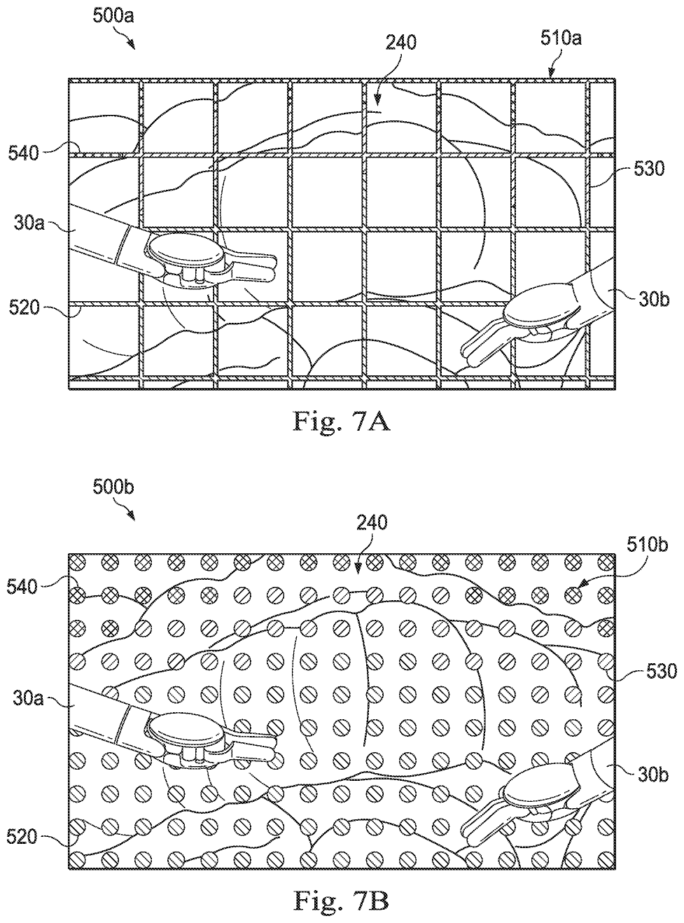

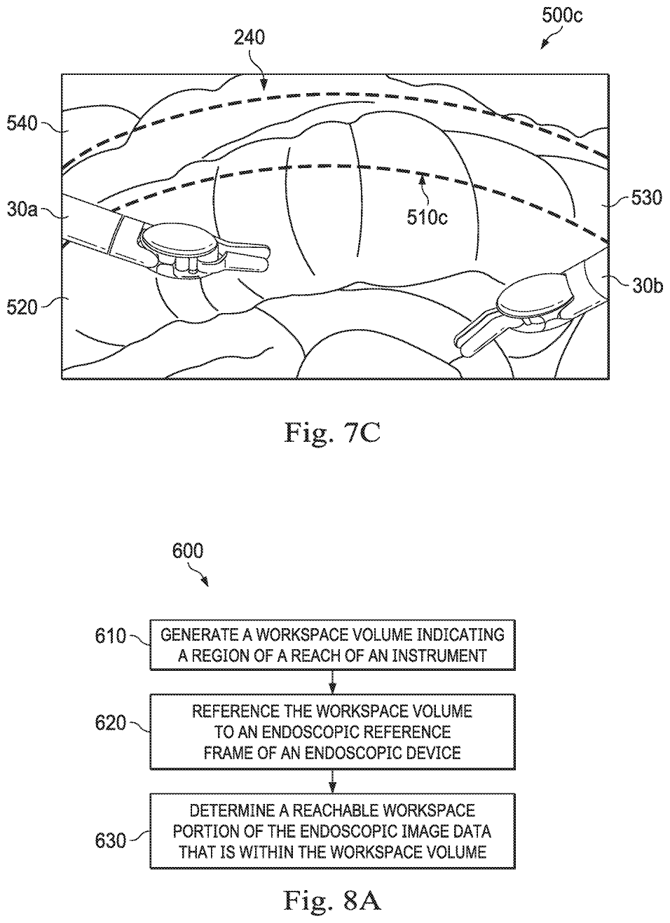

In FIG. 6B, the false graphic 420 occludes the unreachable portion 260, but in other embodiments, other false graphic treatments may be applied that allow the unreachable portion 260 to remain visible but provide visual cues to indicate the limits of the reachable workspace. FIG. 7A is an image 500a of an endoscopic view with a false graphic including a color-coded grid indicating a reachable workspace portion 520 overlaid on a model of the patient anatomy 240 according to some embodiments. The image 500a is an image of the patient anatomy 240 from the endoscopic view. The image 500a includes a false graphic grid overlay 510a, which indicates a reachable workspace 520, a partially-reachable workspace 530, and an unreachable workspace 540. In the embodiment shown in FIG. 7A, the overlay 510a is a color-coded grid. In some embodiments, the lines of the grid may run under/behind the instruments 30a, 30b (as shown in FIG. 7A). In other embodiments, the lines of the grid may run over/in front of the instruments 30a, 30b. In still other embodiments, the instruments 30a, 30b may be masked/hidden/removed from the image 500a. The reachable workspace 520 may be part of the reachable workspace portion 230. In some embodiments, the reachable workspace 520 denotes an area where one or more instruments (e.g., the instruments 28, 30a-c) have full range of motion. In some examples, the partially-reachable workspace 530 denotes an area where the instruments 30a, 30b, for example, can reach, but some of the instruments' motions may be more restricted (i.e., the instruments 30a, 30b may be nearing their kinematic limits). In other embodiments, the unreachable workspace 540 denotes an area where the instruments 30a, 30b cannot reach. The graphic overlay 510a may indicate the reachable workspace 520 with a green color, the partially-reachable workspace 530 with an orange color, and the unreachable workspace 540 with a red color. Each of the workspaces 520, 530, 540 may be identified by any other color. In some embodiments, each of the workspaces 520, 530, 540 may be the same color but may be different shades of that same color. For example, a gray-scale shading scheme may be used. In some embodiments the grid may be formed of tesselated shapes other than squares.

FIG. 7B is an image 500b of an endoscopic view with a false graphic including a pattern of color-coded dots indicating a reachable workspace portion 520 overlaid on a model of the patient anatomy 240 according to some embodiments. The image 500b is an image of the patient anatomy 240 from the endoscopic view. The image 500b includes a false graphic dot pattern overlay 510b, which indicates a reachable workspace 520, a partially-reachable workspace 530, and an unreachable workspace 540. In the embodiment shown in FIG. 7B, the overlay 510b is a grouping of color-coded dots. In some embodiments, the dots may run under/behind the instruments 30a, 30b (as shown in FIG. 7B). In other embodiments, the dots may run over/in front of the instruments 30a, 30b. In still other embodiments, the instruments 30a, 30b may be masked/hidden/removed from the image 500b. The graphic overlay 510b may indicate the reachable workspace 520 with a green color, the partially-reachable workspace 530 with an orange color, and the unreachable workspace 540 with a red color. As discussed above, each of the workspaces 520, 530, 540 may be identified by any other color. In some embodiments, each of the workspaces 520, 530, 540 may be the same color but may be different shades of that same color.

FIG. 7C is an image 500c of an endoscopic view with a false graphic including contour lines indicating a reachable workspace portion 520 overlaid on a model of the patient anatomy 240 according to some embodiments. The image 500c is an image of the patient anatomy 240 from the endoscopic view. The image 500b includes a false graphic contoured line overlay 510c, which indicates a reachable workspace 520, a partially-reachable workspace 530, and an unreachable workspace 540. In the embodiment shown in FIG. 7C, the overlay 510c includes contour lines. As shown in the image 500c, the contour lines are closer together at the boundaries between the reachable workspace 520, the partially-reachable workspace 530, and the unreachable workspace 540. In some embodiments, the contour lines may run under/behind the instruments 30a, 30b (as shown in FIG. 7C). In other embodiments, the contour lines may run over/in front of the instruments 30a, 30b. In still other embodiments, the instruments 30a, 30b may be masked/hidden/removed from the image 500c. In some embodiments, the contour lines may be color-coded in a manner similar to that discussed above.

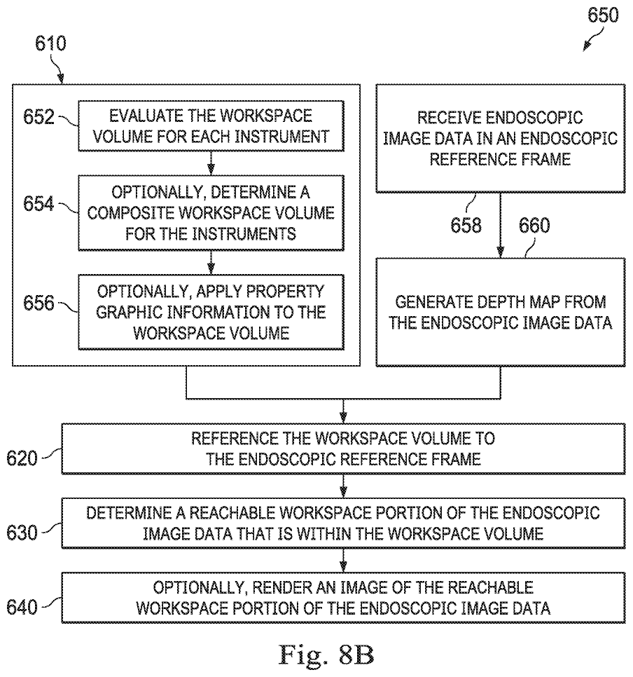

FIG. 8A illustrates a method 600 for generating a workspace volume (e.g., the workspace volume 110) according to some embodiments. The method 600 is illustrated as a set of operations or processes 610 through 630 and is described with continuing reference to FIGS. 1A-7C. Not all of the illustrated processes 610 through 630 may be performed in all embodiments of method 600. Additionally, one or more processes that are not expressly illustrated in FIG. 8A may be included before, after, in between, or as part of the processes 610 through 630. In some embodiments, one or more of the processes 610 through 630 may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes 610 through 630 may be performed by the control system 20.

At a process 610, a workspace volume (e.g., the workspace volume 110) indicating a region of a reach of an instrument (e.g., the instrument 30a) is generated. The workspace volume 110 includes a reachable workspace portion 120, and unreachable portions 130, 140, 150.

At a process 620, the workspace volume is referenced to an endoscopic reference frame of an endoscopic device (e.g., the imaging device 28). The endoscopic device captures endoscopic image data, which may be captured by a left eye camera and a right eye camera of the imaging device 28. In some embodiments, the captured endoscopic image data is stored in the memory 24 of the control system 20.

At a process 630, a reachable workspace portion (e.g., the reachable workspace portion 120) of the endoscopic image data that is within the workspace volume is determined. In some embodiments, the reachable workspace portion of the endoscopic image data is determined by analyzing the endoscopic image data to generate a dense disparity map that spatially relates the endoscopic image data between a left eye of the endoscope, which may include left eye image data, and a right eye of the endoscope, which may include right eye image data. In such embodiments, the reachable workspace portion may further be determined by converting the dense disparity map to a depth buffer image (e.g., the depth buffer image 320). Further detail is provided at FIG. 8B.

In some embodiments, the method 600 may further include the process of determining an unreachable portion of the endoscopic image data that is outside of the workspace volume 110. In some examples, the method 600 may further include the process of displaying the reachable workspace portion 120 of the endoscopic image data without the unreachable portion of the endoscopic image data. In some embodiments, the endoscopic image data and the reachable workspace portion 120 may be displayed on a display screen of one or more systems of the auxiliary systems 26. In some embodiments, the method 600 may further include the process of rendering a composite image including a false graphic and an endoscopic image of the patient anatomy.

FIG. 8B illustrates a method 650 for generating a workspace volume (e.g., the workspace volume 110) according to some embodiments. The method 650 includes the processes 610-630 and includes additional detail that may be used to perform the processes 610-630. Not all of the illustrated processes may be performed in all embodiments of method 650. Additionally, one or more processes that are not expressly illustrated in FIG. 8B may be included before, after, in between, or as part of the illustrated processes. In some embodiments, one or more of the processes may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes may be performed by the control system 20.

The process 610 of generating a workspace volume may include the process 652 of evaluating the workspace volume for each instrument. The workspace volumes or optionally just the reachable workspace portions may be transformed into a common coordinate system. The process 610 may also, optionally, include a process 654 of determining a composite workspace volume or a composite of the reachable workspace portions for the set of instruments. The composite workspace volume may be transformed into an endoscopic reference frame. The process 610 may also, optionally, include a process 656 of applying graphical information to the workspace volume. The graphical information may include patterns, tesselations, colors, saturations, illuminations or other visual cues to indicate regions that are reachable, partially reachable, or unreachable by one or more of the instruments.

At a process 658, captured endoscopic image data in the endoscopic reference frame may be received. At a process 660, a depth mapping procedure may be performed. This process may be performed by the control system 20 and/or one or more systems of the auxiliary systems 26. For clarity of discussion, the following discussion will be made with reference to the control system 20. In some examples, the control system 20 analyzes endoscopic image data (which may be captured by the imaging device 28) and generates a dense disparity map for a set of data captured by the left-eye camera and for a set of data captured by the right-eye camera. These sets of data are part of the captured endoscopic image data discussed above. The control system 20 then converts the dense disparity map to a depth buffer image (e.g., the depth buffer image 320). The depth buffer image 320 may be generated in the endoscopic reference frame. Based on the depth buffer image 320, the control system 20 determines which portion(s) of the patient anatomy 240 are within the reachable workspace portion 230 of the composite workspace volume 220, which has been referenced to the endoscopic reference frame. In some embodiments, the control system 20 may render the left eye image 300 of the reachable workspace portion 230 (which may be a reachable workspace portion of endoscopic image data). Additionally, the control system 20 may render the right eye image 310 of the reachable workspace portion 230 to generate a composite image (e.g., the reconstructed 3D image 330) of the reachable workspace portion 230. In several examples, the control system 20 may reference the workspace volume 110 and/or the composite workspace volume 220 to an endoscopic reference frame of an endoscopic device (e.g., the imaging device 28). Depth mapping is described in further detail, for example, in U.S. Pat. App. Pub. No. 2017/0188011, filed Sep. 28, 2016, disclosing “Quantitative Three-Dimensional Imaging of Surgical Scenes,” and in U.S. Pat. No. 8,902,321, filed Sep. 29, 2010, disclosing “Capturing and Processing of Images Using Monolithic Camera Array with Heterogeneous Imagers,” which are both incorporated by reference herein in their entirety.

In some embodiments, the depth buffer image 320 can be loaded as a buffer, such as a Z-buffer, and the depth buffer image 320 may be used to provide depth occlusion culling of the rendered left eye image 300 and the rendered right eye image 310. This allows for the control system 20 to cull the rendered left eye image 300 and the rendered right eye image 310 using the reachable workspace portion 230.

To achieve the depth occlusion culling, the control system 20 may render the left eye image 300 and the right eye image 310 with the reachable workspace portion 230, which has been referenced to the endoscopic reference frame at process 620. At the process 630, the reachable workspace portion of the endoscopic image data that is within the workspace volume is determined. In some examples, the control system 20 combines the reachable workspace portion 230 and the reconstructed 3D image 330. The reachable workspace portion 230 acts a buffer, and in some embodiments, only pixels of the model of the patient anatomy 240 within the reachable workspace portion 230 are displayed in the reconstructed 3D image 330. In other embodiments, only pixels of the patient anatomy 240 within the reachable workspace portion 230, within the view of the imaging device 28, and closer to the imaging device 28 that other background pixels are displayed in the reconstructed 3D image 330. In other embodiments, the control system 20 overlays the reachable workspace portion 230 on the reconstructed 3D image 330. At a process 640, optionally the composite image of the reachable workspace portion 230 and the endoscopic image data 330 is rendered on a display.

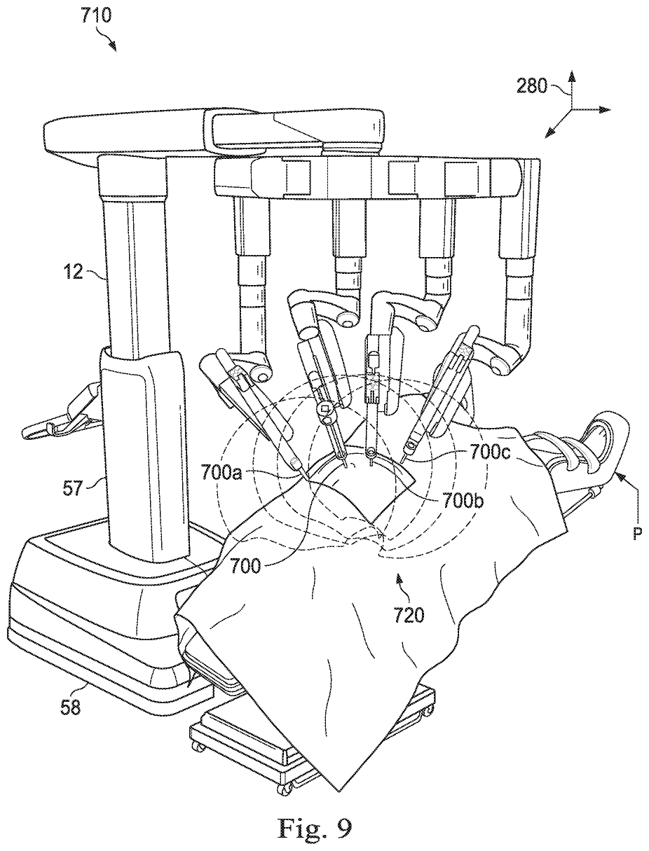

FIG. 9 is a perspective view of a system workspace 710 in which the patient P (which includes patient anatomy 240) and the assembly 12 are located. In the embodiment shown in FIG. 9, each arm 54 of the assembly 12 includes a blunt cannula 700, 700a, 700b, 700c. Each blunt cannula represents a working cannula (which may be a surgical cannula) through which each instrument 28, 30a-c may be inserted to enter the patient anatomy. For example, the blunt cannula 700 corresponds to a surgical cannula for receiving the imaging device 28. The blunt cannula 700a corresponds to a surgical cannula for receiving the surgical tool 30a. The blunt cannula 700b corresponds to a surgical cannula for receiving the surgical tool 30b. The blunt cannula 700c corresponds to a surgical cannula for receiving the surgical tool 30c. The blunt cannulas 700, 700a-c may allow the surgeon S to determine the ideal placement for the working cannulas for each instrument 28, 30a-c prior to making any incisions in the patient P. In several embodiments, the surgeon S can determine the ideal cannula placement by determining the location of a workspace volume for each blunt cannula 700, 700a-c corresponding to the cannulas for each instrument 28, 30a-c. Therefore, the surgeon S can place the arms 54 in the ideal position to perform the surgical procedure without making unnecessary incisions in the patient P. This allows the surgeon to place the instruments 28, 30a-c at ideal incision locations to perform the surgical procedure. In several examples, the surgeon S may analyze the workspace volumes for each blunt cannula 700, 700a-c to determine how to position the arms 54 to ensure that the composite reachable workspace portion (e.g., the reachable workspace portion 230) includes as much of the patient anatomy 240 as possible. In some embodiments, the workspace volumes for each blunt cannula 700, 700a-c may be displayed on the display system 35 and/or on a display screen of one or more systems of the auxiliary systems 26 before the instruments 28, 30a-c are installed on their corresponding arms 51. In such embodiments, the surgeon S can visualize the reachable workspace portion 230 in the endoscopic view while the surgeon S or an assistant adjusts one or more of the arms 54 and/or the arms 51 to affect the placement of one or more of the blunt cannulas 700, 700a-c.

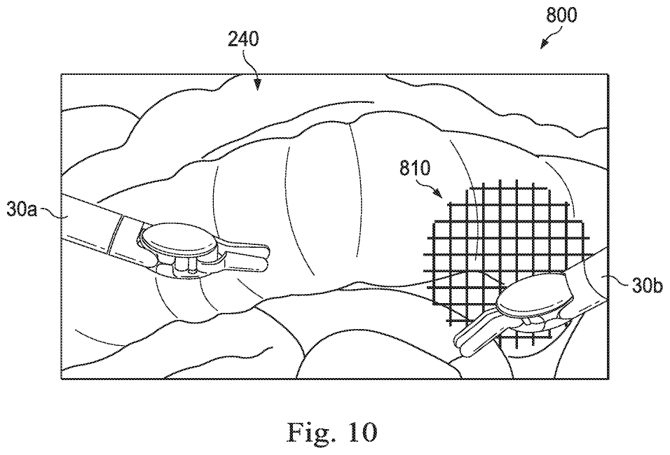

FIG. 10 is an image 800 of an endoscopic view with a three-dimensional surface patch 810 overlaid on a model of the patient anatomy 240 according to some embodiments. The image 800 includes a rendered image of the patient anatomy 240, a rendered image of the instruments 30a, 30b, and a surface patch 810. In some embodiments, the surface patch 810 is used to portray the reachable workspace portion for each surgical tool 30a-c. In some examples, the surface patch 810 is a 3D surface patch that portrays position and orientation of restricted motion of a tip of the instrument 30b, for example. While the discussion below will be made with reference to instrument 30b, it is to be understood that the surface patch 810 can be depicted for any one or more of the instruments 30a-c.

In several embodiments, the surface patch 810 is displayed in the image 800 when motion of a tip of the instrument 30b is limited, such as when the instrument 30b is nearing or has reached one or more of its kinematic limits. The surface patch 810 portrays the surface position and orientation of the restricted motion of the instrument 30b. In some embodiments, the surgeon S perceives kinematic limits of the instrument 30b via force feedback applied to the input control devices 36. The force feedback may be the result of forces due to kinematic limits of the instrument 30b itself, interaction between the instrument 30b and the patient anatomy 240, or a combination thereof. In some examples, the surface patch 810 is displayed in the image 800 when the force feedback is solely the result of forces due to kinematic limits of the instrument 30b. In other examples, the surface patch 810 may be displayed in the image 800 when the force feedback is solely the result of forces due to interaction between the instrument 30b and the patient anatomy 240.

One or more elements in embodiments of this disclosure may be implemented in software to execute on a processor of a computer system such as a control processing system. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus, and various systems may be used with programs in accordance with the teachings herein. The required structure for a variety of the systems discussed above will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A method comprising:
generating a workspace volume indicating an operational region of reach;
referencing the workspace volume to an image capture reference frame of an image capture device, wherein the image capture device captures image data; and
determining a reachable workspace portion of the image data that is within the workspace volume.

2. The method of claim 1, wherein the operational region of reach includes a region of a reach of an instrument.

3. The method of claim 1, wherein the operational region of reach includes a region of a reach of an arm of a manipulating system, the arm being coupled to an instrument.

4. The method of claim 1, further comprising:
determining an unreachable portion of the image data that is outside of the workspace volume.

5. The method of claim 4, further comprising:
displaying the reachable workspace portion of the image data without the unreachable portion of the image data; and
displaying a false graphic in place of the unreachable portion of the image data.

6. (canceled)

7. The method of claim 5, wherein the false graphic comprises at least one of a color hue, a color saturation, an illumination, or a surface pattern.

8. The method of claim 4, further comprising:
displaying the reachable workspace portion of the image data and the unreachable portion of the image data, wherein the unreachable portion is modified by a false graphic.

9. (canceled)

10. The method of claim 4, further comprising:
displaying the reachable workspace portion of the image data and the unreachable portion of the image data; and
displaying an overlay on the image data.

11. (canceled)

12. The method of claim 10, wherein the overlay comprises at least one of a colored grid, a plurality of colored dots, or a plurality of contour lines.

13. The method of claim 1, wherein the workspace volume has a generally spherical shape, and wherein a radius of the generally spherical shape is determined based on an insertion range of an instrument.

14. (canceled)

15. The method of claim 1, wherein determining the reachable workspace portion comprises:
analyzing the image data to generate a dense disparity map for a set of left eye image data of the image data and a set of right eye image data of the image data; and
converting the dense disparity map to a depth buffer image, wherein the reachable workspace portion of the image data is determined from the depth buffer image.

16. The method of claim 15, further comprising:
rendering a left eye image of the reachable workspace portion of the image data;
rendering a right eye image of the reachable workspace portion of the image data; and
generating a composite image of the reachable workspace portion of the image data.

17. The method of claim 1, further comprising:
generating a second workspace volume indicating a region of a reach of a second instrument;
referencing the second workspace volume to the image capture reference frame;
generating a composite workspace volume by combining the workspace volume and the second workspace volume;
referencing the composite workspace volume to the image capture reference frame; and
determining a reachable workspace portion of the image data that is within the composite workspace volume.

18. The method of claim 17, wherein generating the composite workspace volume comprises:
determining that an arm coupled to an instrument will contact a second arm coupled to a second instrument during a surgical procedure;
based on the determined contact, computing a distance field for the instrument and computing a second distance field for the second instrument; and
based on the computed distance field, determining a volumetric distance field.

19. The method of claim 18, further comprising:
determining an unreachable portion of the image data that is outside of the composite workspace volume; and
displaying the volumetric distance field as a false graphic in place of the unreachable portion of the image data.

20. The method of claim 18, wherein computing the distance field for the instrument comprises:
determining a closest distance between a surface of the arm and a surface of the second arm.

21-35. (canceled)

36. A method comprising:
generating a workspace volume indicating an operational region of reach;
referencing the workspace volume to an image capture reference frame of an image capture device, wherein the image capture device captures image data;
determining a reachable workspace portion of the image data that is within the workspace volume; and
based on the determined reachable workspace portion, determining an incision location of an instrument.

37. The method of claim 36, wherein the operational region of reach includes a region of a reach of the instrument.

38. (canceled)

39. The method of claim 36, wherein the workspace volume is generated prior to a beginning of a medical procedure.

40. The method of claim 36, further comprising:
determining an unreachable portion of the image data that is outside of the workspace volume;
displaying the reachable workspace portion of the image data without the unreachable portion of the image data; and
displaying a false graphic in place of the unreachable portion of the image data, wherein the false graphic comprises at least one of a color hue, a color saturation, an illumination, or a surface pattern.

41-61. (canceled)
